(19) United States
(12) Patent Application Publication
(10) Pub. No.: US 2008/0200954 A1
(43) Pub. Date: Aug. 21, 2008

(54) SKULL CLOSURE DEVICE AND METHOD

(76) Inventor: Keith A. Tucci, Greenville, NC (US)

Correspondence Address: WARD AND SMITH, P.A., 1001 COLLEGE COURT, P.O. BOX 867, NEW BERN, NC, 28563-0867, US

(21) Appl. No.: 12/033,815

(22) Filed: Feb. 19, 2008

Related U.S. Application Data
(60) Provisional application No. 60/890,623, filed on Feb. 20, 2007.

(51) Int. Cl. A61B 17/58; A61B 17/00

(52) U.S. Cl. 606/280; 606/151

(57) ABSTRACT

A fastening system and method includes a deformable connecting member configured for being connected at one end to a skull flap of a patient who has undergone a craniectomy procedure, and at the other end to the skull of the patient. In this manner, the skull flap is attached to the skull in a manner allowing limited movement of the skull flap. This allows for relief of intra-cranial pressures for patients having undergone a craniectomy procedure. The connecting members are made of a material which is biocompatible with the patient.

### CROSS-REFERENCE TO RELATED APPLICATIONS

This Application is related to and claims priority to Provisional Application No. 60/890,623 filed Feb. 20, 2007 of the same inventor, the disclosure of which is specifically incorporated by reference herein.

### FIELD OF THE INVENTION

The present invention relates generally to a fastening system, particularly for use in reattaching a skull flap, removed during brain surgery, and to a method of reattaching the skull flap using the fastening system.

### BACKGROUND OF THE INVENTION

Decompressive craniectomy is a procedure that has been used regularly in the past for traumatic and medical elevations of intra-cranial pressures (“ICP”) due to traumatic brain injury. While in the past the technique resulted in improved survival, it also resulted in a disappointing lack of neurologic recovery. More recently, there have been developed a number of damage control techniques resulting in a resurgence of interest in the use of decompressive craniectomy in the case of traumatic brain injury as a damage control method for the brain.

Decompressive craniectomy, however, is not a procedure which should be conducted without serious evaluation and consideration of potential complicating factors. More specifically, post-operative complications, while not common, may be quite severe. Among such complications are hematoma, incisional CSF drainage, encephalomyelocele, traumatic epilepsy, dilation and/or migration of lateral ventricle under cranial defect hydrocephalous VP shunt, subdural fluid collections, meningitis, and acosmetic concavity bone defect.

In the past, after the initial decompressive operation was conducted, the bone flap or skull flap which was removed was stored. In one storage technique, the bone flap was kept in a bone freezer for up to a year for future reimplantation. In an alternate mode of storage, the flap was implanted in the abdominal fat of the patient for preservation.

In the typical case scenario, cranioplasty for replacement of the skull flap or prosthetic materials was completed between three and six months after the flap removal. During that time, patients were without a cranial flap, typically for several months, which placed them at risk of injury to the unprotected brain. During this time period, rehabilitation was required and balance was poor, resulting in the risk of falling and damage to the brain.

As a follow up procedure, the skull was reconstructed, typically with the original bone flap. However complications often arose because stored bone flaps are exposed to contamination, and bone resorption may occur in the case of abdominal fat stored bone flaps.

Other complications may arise from changes in the flap during storage, complicating reattachment of the skull flap at a later time. In practice, it is desirable to attach the side furthest from the temporal lobes to avoid the high lethality of compression in the region of the temporal lobes.

Thus, it becomes desirable to provide a system and method avoiding the complications of having a patient with an unprotected brain region following craniectomy while at the same time allowing relief of intra-cranial pressures resulting from brain injury.

### SUMMARY OF THE INVENTION

The present invention provides for use of a connecting member attachable to both the skull and skull flap (also known alternatively as a cranial flap or bone flap) resulting from a craniectomy procedure. The connecting member is deformable in the sense that it can act as a hinge such that the scalp can be closed after the craniectomy and the skull flap allowed to settle back to its proper position upon subsiding of ICP. The cranial flap is thus allowed to open and close with ICP, such that ICP can be reduced while the patient skull flap is left in situ. In this manner, necessary brain protection is provided and a second operative procedure which was in the past generally needed to reconstruct the skull is avoided.

In one aspect, the fastening system includes a deformable connecting member configured for being connected at least at one end to a skull flap which has been created from a decompressive craniectomy procedure. The connecting member is connected at least at the other end to the skull in a manner allowing limited movement of the skull flap when installed in a patient's skull, thereby accommodating relief of intra-cranial pressures. The deformable connecting member is made of a material which is biocompatible with the patient. Openings are provided at each end of deformable connecting member and serve to receive securing elements to secure the deformable connecting member at each respective end to a patient's skull and to a patient's skull flap in a manner covering the brain region from where the skull flap was removed.

Embodiments may include the connecting members as an elongate member including a hinge for allowing the bone flap to move outwardly from the brain (such as in the case of elevated ICP). Alternatively, the deformable connecting member may be of flexible material. In the case of a hinge, it may also be spring loaded for displacing the skull flap towards the brain upon reduction of ICP. A stop may be provided separate from the elongate member for attachment to the skull or the skull flap to prevent the skull flap from receding downwardly beyond a desired position into the patient's brain.

Another aspect of the invention includes a method of reattaching a skull flap removed during brain surgery and using the aforementioned fastening system. The method includes attaching one end of the connecting member to a skull flap at one end thereof and attaching the other end to a patient's skull in a manner in which the skull flap fits into the skull opening which resulted from removal of the skull flap.

Another embodiment involves a two part slide secured to the skull flap and a patient's skull to allow sliding movement between pairs of slides to allow the skull flap to rise above the level of the skull to relieve ICP.

These and other advantages and features that characterize the invention are set forth in the claims annexed hereto and forming a further part hereof. However, for a better understanding of the invention, and of the advantages and objects attained through its use, reference should be made to the Drawings and to the accompanying descriptive matter, in which there are described exemplary embodiments of the invention.

### BRIEF DESCRIPTION OF THE DRAWINGS

FIG. 1 is a side, partial cross section view of the head of a patient having undergone a decompressive craniectomy procedure with the skull flap attached with the system in accordance with the invention.

FIG. 2 is a top plan view of a patient's skull with the skull flap attached in accordance with the system of the invention.

FIG. 3 is a top plan view of one embodiment of a connecting member including a hinge.

FIG. 4 is a side view of the embodiment of FIG. 3.

FIG. 5 is a top plan view of an alternative embodiment of a connecting member including a hinge in accordance with the invention.

FIG. 6 is a side view of the embodiment of FIG. 5.

FIGS. 7-9 illustrate in top plan view three alternative embodiments of a hinged connecting member in accordance with the invention.

FIG. 10 is a side view of a deformable connecting member which deforms by bending.

FIG. 11 is a side view in partial cross section of an alternative skull flap attachment system in accordance with the invention.

### DETAILED DESCRIPTION

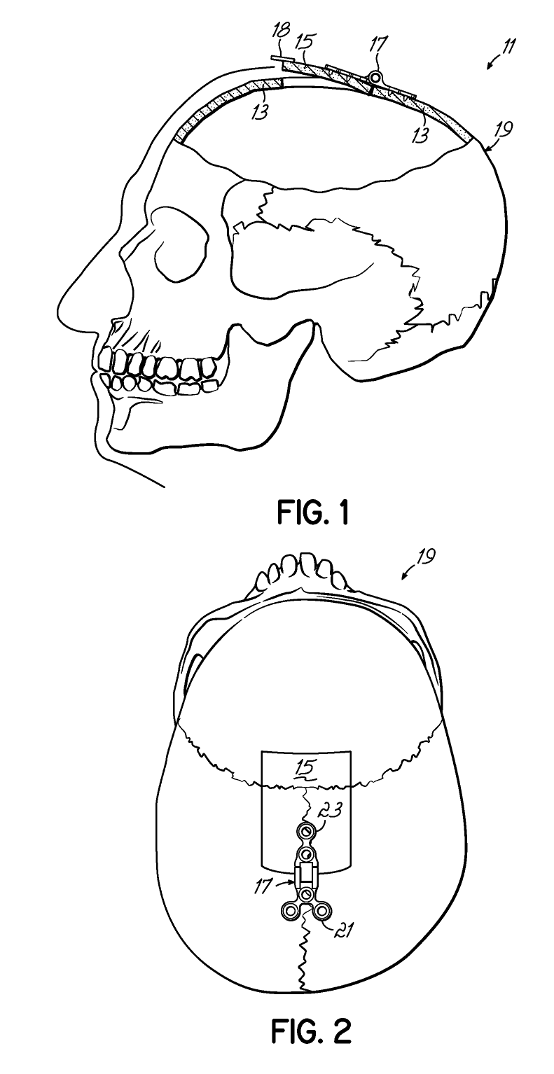

FIG. 1 is a side partial cross section view of a patient's head 11 having undergone a craniectomy. The skull flap 15 has been attached with the system in accordance with the invention. More specifically, the patient's head 11 includes the skull 19 having skull portions 13 and an opening which resulted from a skull flap 15 having been removed therefrom. The skull flap 15 is placed to cover the opening resulting from a craniectomy procedure through means of a connecting member 17. The connecting member 17 may be a hinged member which attaches at one end to a skull portion 13 and at the other end to the skull flap 15. As a result of the hinge arrangement, intra-cranial pressure can be relieved by allowing the skull flap 15 to expand away from a patient's brain. A separate stop member 18 can also be mounted on the skull or flap to limit movement of the skull flap 15 into the brain cavity, as shown, for example, mounted on the top of skull flap 15 for stopping engagement with skull portion 13 as skull flap 15 recedes towards the patient's brain. While FIG. 1 shows only a single connecting member 17, as will be readily apparent to those of ordinary skill, more than one connecting member 17 can be attached as necessary for the intended function, and depending on the size of the skull flap 15.

FIG. 2 illustrates a patient's skull 19 in top plan view. The skull 19 includes a skull flap 15 which has been placed back in an opening created by a craniectomy procedure. The skull flap 15 is held by means of a connecting member 17 which is hinged. In an exemplary embodiment the member 17 includes a first end 21 with three openings, for example, for screws for attachment to the patient's skull 19. A second end 23 is secured to the skull flap 15 and includes two openings for receiving, for example, securing screws therein.

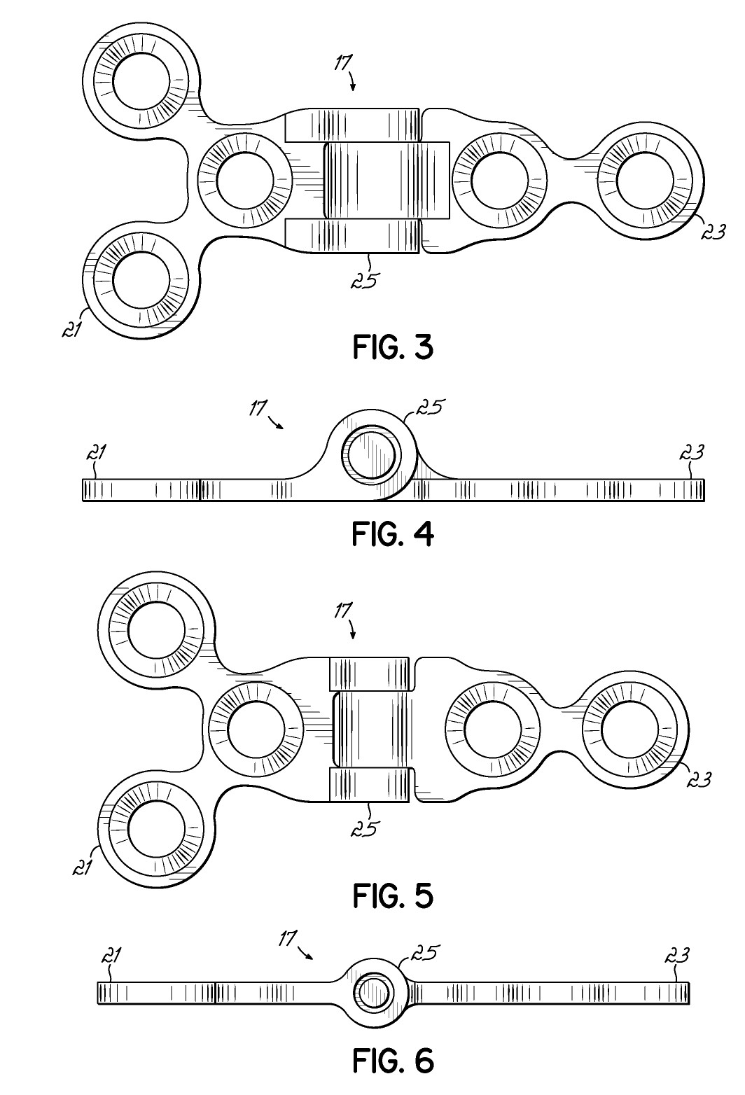

FIGS. 3 and 4 illustrate one embodiment of a hinged connecting member in accordance with the invention. The connecting member 17 is elongate in shape and includes a central hinge region 25 and ends 21 and 23. First end 21, for example, can be configured in a triangle form with openings to provide a stable and secure attachment through, for example, the use of screws or other pin like connectors to a patient's skull. The second end 23 serves to be secured to the skull flap, for example, through two longitudinally aligned connecting holes. As may be appreciated from FIG. 4, the bottom of the connecting member 17 is flat with the profile of the hinge extending upwardly. The connection member 17 is sized for use as described, in securing a skull flap 15.

FIGS. 5 and 6 illustrate an alternative embodiment of the connecting member 17 of the invention. While similar in shape and arrangement to the embodiment of FIGS. 3 and 4. In this case the hinge 25 projects both upwardly and downwardly and is not flat at the bottom. A small hole may be burred out of the skull and skull flap for the hinge 25 to sit in the craniectomy site. In this particular embodiment, the projection upwardly beneath the patient's skin resulting from hinge 25 would be minimized when compared to the embodiment of FIGS. 3 and 4.

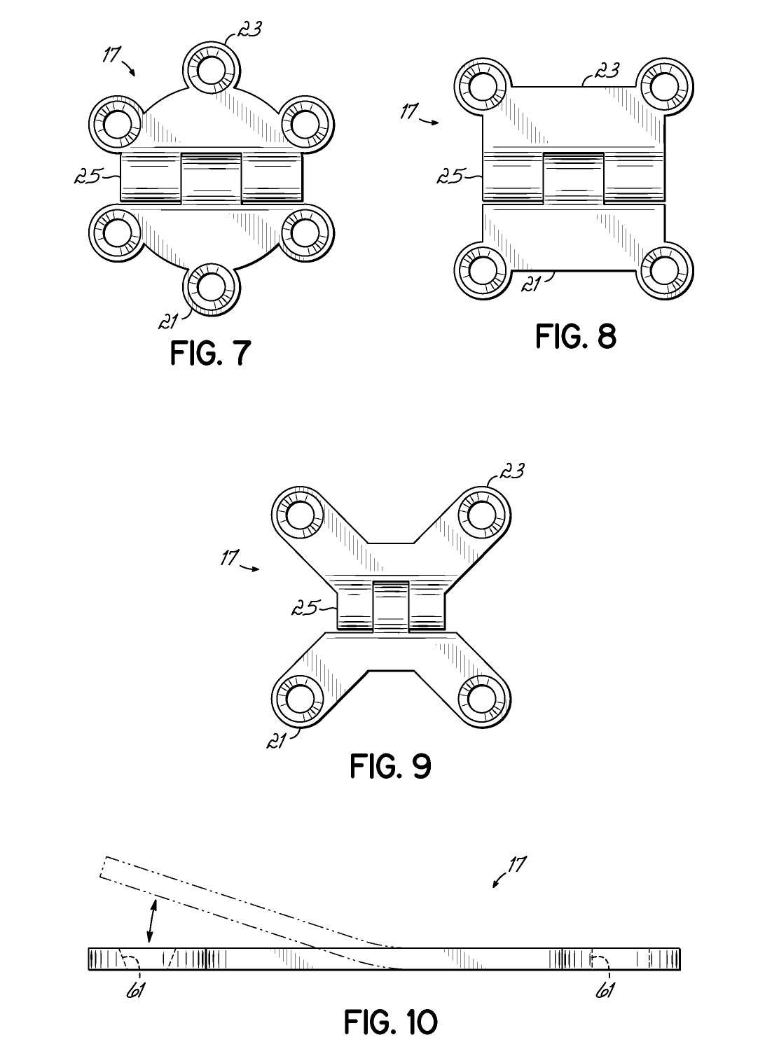

Yet still other embodiments in accordance with the invention are shown in FIGS. 7-9 and can take the shape of a general circular member as shown in FIG. 7, or a rectangular member as shown in FIG. 8, or even in a generally star shaped configuration as shown in FIG. 9. In all three embodiments there is provided a first end 21, a second end 23, and a hinge region 25 for the connecting member 17.

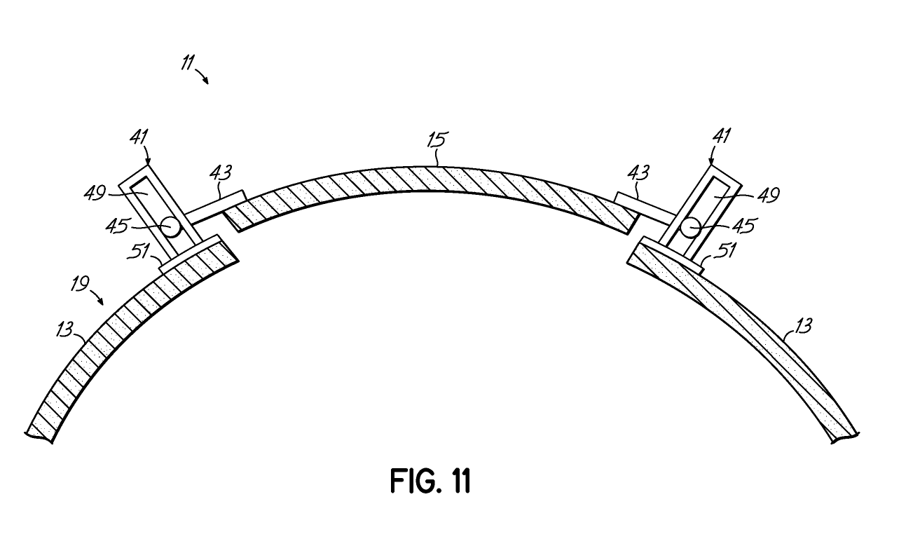

Although the invention has been described with respect to hinged elements, it will be readily apparent to those of ordinary skill in the art that the hinge can be eliminated and the connecting member 17 can be made of flexible material which would allow the skull flap to move upwardly to relieve intra-cranial pressures. This is shown in exemplary form in FIG. 10. The bending of the member 17 is shown in dashed line and showing openings 61 therein for receiving securing members such as screws.

When hinges are used, the hinges may be spring loaded so that as intra-cranial pressures recede, the spring force of the hinge forces the skull flap downwardly to settle into the skull opening. This assists the skull flap 15 settling into the skull opening as ICP is reduced.

In yet still another alternative embodiment in accordance with the invention, instead of connecting the skull flap at only one end, a slide mechanism can be provided as illustrated in FIG. 11. The skull flap 15 includes an attached elongate member 43 having an end 45 to be engaged within a slide region 49 of a slide member 41. A slide member 41 includes a base 51 for attachment to the skull portion 13 of a patient's head 11 and the arrangement is connected at both ends of the skull flap 15 through the slide mechanism. As may be appreciated, upon intra-cranial pressures increasing the end 45 of connecting member 43 is allowed to slide upwardly in slide region 49 while retained by the walls 47 of the slide member 41. Conversely, upon a decrease in intra-cranial pressure, the skull flap 15 can recede downwardly towards the patient's brain.

According to a method of use of the system of the invention connecting members substantially as previously described are provided. One end of the connecting member may be attached to the skull flap at one end thereof, and the other end attached to the skull in a manner wherein the skull flap fits into a skull opening which resulted from removal of the skull flap. In terms of the materials selected for the connecting members, it is only required that they be biocompatible with the patient and sufficiently rigid to withstand use over long periods of time and not degrade when installed in the patient's skull region. Examples of such materials include stainless steel, titanium and other like metals. Alternatively, certain deformable plastics may also be used to provide such a member in an embodiment not employing a hinge.

While the present invention has been illustrated by a description of various embodiments and while these embodiments have been described in considerable detail, it is not the intention of the Applicant to restrict, or any way limit the scope of the appended claims to such detail. The invention in its broader aspects is therefore not limited to the specific details, representative apparatus and method, and illustrative example shown and described. Accordingly, departures may be made from such details without departing from the spirit or scope of Applicant's general inventive concept.

What is claimed is:

1. A fastening system for a skull flap, comprising:
a deformable connecting member configured of size and shape for being connected at least at one end to a skull flap resulting from a decompressive craniectomy procedure, and at least at the other end to a skull in a manner allowing limited movement of the skull flap when installed in a patient's skull;
said deformable connecting member being made of a material which is bio-compatible with a patient; and
connecting means at each end of the deformable connecting member for securing the connecting member at a respective end to a patient's skull and to a patient's skull flap in a manner covering the brain region from where the skull flap was removed.

2. The fastening system of claim 1, wherein said deformable connecting member further comprises a hinge for allowing the bone flap to move outwardly away from the brain when installed.

3. The fastening system of claim 1, wherein said deformable connecting member is of flexible material for allowing the skull flap to move outwardly from the brain.

4. The fastening system of claim 1, wherein said deformable connecting member is made of stainless steel.

5. The fastening system of claim 1, further comprising an opening at one end of the connecting member for attachment through fastening elements to a patient's skull and an opening at the other end for attachment to a patient's skull flap through fastening elements.

6. The fastening system of claim 2, wherein said hinge is spring loaded for displacing the skull flap towards the brain as intra-cranial pressures ease when said fastening system is installed.

7. The fastening system of claim 1, further comprising a stop member attachable to one of a patient's skull or skull flap for preventing the bone flap from descending into the brain as intra-cranial pressures ease when said fastening system is installed.

8. The fastening system of claim 7, wherein said stop member is rigid and is of a material which is biocompatible with a patient's skull and skull flap.

9. The fastening system of claim 2, wherein said hinged connecting member comprises attachment openings at each end thereof for allowing securing members to pass therethrough for securing said hinge to said skull and skull flap.

10. A method of reattaching a skull flap, removed during brain surgery, and using a fastening system comprising a deformable connecting member, the method comprising: attaching one end of the connecting member to the skull flap at one end thereof, and attaching the other end of said connecting member to a patient's skull in a manner wherein the skull flap fits into a skull opening which resulted from removal of the skull flap.

11. The method of claim 10, wherein said deformable connecting member comprises a hinge for allowing the skull flap to move outwardly away from a patient's brain.

12. The method of claim 10, wherein said deformable connecting member is of flexible material for allowing the skull flap to move outwardly from a patient's brain.

13. The method of claim 10, wherein said deformable connecting member is made of stainless steel.

14. The method of claim 10, wherein said deformable connecting member comprises an opening at one end thereof for attachment through fastening elements to a patient's skull and an opening at the other end for attachment to a patient's skull flap.

15. The method of claim 11, wherein said hinge is spring loaded for displacing the skull flap towards the brain as intra-cranial pressures ease when said fastening system is installed.

16. The method of claim 10, further comprising attaching a stop member to at least one of the skull flap and a patient's skull in a manner to prevent the skull flap from settling beyond the level of a patient's skull.

17. The method of claim 16, wherein said stop member is separate from said deformable connecting member.

18. The method of claim 17, wherein said stop member is rigid and is of a material which is biocompatible with a patient's skull and skull flap.

19. The method of claim 11, wherein said hinged connecting member comprises attachment opening at end thereof for allowing securing members to pass therethrough for securing said hinged connecting member to said skull and skull flap.

20. A fastening system, comprising:
a first member for being secured to one of a skull flap and a skull and having a sliding engagement extension; and
a second member configured for receiving said sliding engagement extension in a slide region thereof, said second member being securable to the other of a patient's skull or skull flap.
